(19) United States
(12) Patent Application Publication
(10) Pub. No.: US 2026/0168031 A1
(43) Pub. Date: Jun. 18, 2026

(54) DOUBLE-STRANDED RNA EXPRESSION SIGNATURES TO PREDICT RESPONSE TO CANCER THERAPY

(71) Applicant: The Regents of the University of California, Oakland, CA (US)

(72) Inventors: Xinshu Xiao, Los Angeles, CA (US); Tracey Chan, Los Gatos, CA (US); Ting Fu, Los Angeles, CA (US)

(73) Assignee: The Regents of the University of California, Oakland, CA (US)

(21) Appl. No.: 19/127,255

(22) PCT Filed: Nov. 7, 2023
(86) PCT No.: PCT/US2023/078955

§ 371 Date: May 5, 2025

Related U.S. Application Data
(60) Provisional application No. 63/424,351, filed on Nov. 10, 2022.

(51) Int. Cl. C12Q 1/6886

(52) U.S. Cl. CPC C12Q 1/6886; C12Q 2600/106

(57) ABSTRACT

In recent years, the diversity of proteins and complexity of gene regulation associated with RNA editing dysregulation has been increasingly appreciated in oncology. By profiling editing in single cells from lung cancer biopsies, we found that the increased editing trend of bulk lung tumors was unique to cancer cells. Elevated editing levels were observed in cancer cells resistant to targeted therapy, and editing sites associated with drug response were enriched. Consistent with the regulation of antiviral pathways by RNA editing, we observed that higher editing levels in cancer cells were associated with reduced anti-tumor innate immune response. In addition, the level of RNA editing in cancer cells was positively associated with somatic point mutation burden. Building upon these discoveries, we have developed new methods and materials that can be used to identify RNA mutations that correlate with tumor mutation burden and cancer innate immunity.

### CROSS REFERENCE TO RELATED APPLICATIONS

This application claims the benefit under 35 U.S.C. Section 119(e) of co-pending and commonly-assigned U.S. Provisional Patent Application No. 63/424,351, filed Nov. 10, 2022, entitled “DOUBLE-STRANDED RNA EXPRESSION SIGNATURES TO PREDICT RESPONSE TO CANCER THERAPY”, which application is incorporated by reference herein.

### STATEMENT REGARDING FEDERALLY SPONSORED RESEARCH AND DEVELOPMENT

This invention was made with government support under CA262686 awarded by the National Institutes of Health. The government has certain rights in the invention.

### TECHNICAL FIELD

Embodiments of the disclosure concern at least the fields of immunology, cell biology, molecular biology, and medicine.

### BACKGROUND OF THE INVENTION

Amongst the various types of RNA editing in the human transcriptome, the most prevalent is A-to-I editing, i.e., deamination of adenosine (A) to inosine (1)¹. Proteins of the adenosine deaminases acting on RNA (ADAR) family, specifically ADAR1 and ADAR2, catalyze this conversion. Facilitated by advances in genomic technologies, recent studies have highlighted the multi-faceted roles of ADAR activity in cancer-.

Specific editing sites have been discovered to transform cancer cell behavior, as well as impact the anti-tumor immune response. Edited at higher levels in multiple cancer types, a recoding site in AZIN1 increases cell growth and invasion by heightening the protein's affinity to antizyme and consequently preventing antizyme-dependent degradation of two oncoproteins^{2,6,7}. In other examples, translation of mRNAs containing recoding events may generate tumor-associated edited peptides, which may prompt anti-tumor T cell responses specific to these editing-derived antigens^{8,9}. Besides protein sequence alteration⁴, editing can affect transcript stability of genes that drive cancer initiation, progression and response to therapy^{2,10}. For example, a FAK-stabilizing intronic site enables a migratory and invasive phenotype in lung adenocarcinoma (LUAD) cells¹¹. RNA editing may regulate mRNA degradation by altering miRNA biogenesis, targeting, or binding^{2,4,12,13}.

Demonstrating both oncogenic and tumor-suppressive capacities, RNA editing has shown potential for clinical applications. In mouse models, combining ADAR deficiency with immune checkpoint blockade (ICB) or DNA methyltransferase inhibitor (DNMTi) therapy improved treatment efficacy through induced interferon (IFN) signaling^{14,15}. For particular cancer cells with pre-existing expression of interferon stimulated genes (ISGs), ADAR loss alone caused cell lethality^{16,17}. On the level of site-specific editing, recoding events individually affected drug sensitivity of two cell lines¹⁸.

Given the significant implications of RNA editing for cancer etiology, it is critical to achieve a deep understanding of tumor-specific RNA editing aberrations and their associated mechanisms. Global profiling of the RNA editomes in tumors is the first step towards this goal: altered editing profiles have been reported in tumors of many cancer types¹⁸⁻²⁰. The functional implications of most of these editing changes, especially those in noncoding regions, are unknown. One crucial role of editing in normal cells is to modify endogenous double-stranded RNAs (dsRNAs), likely altering their secondary structures to prevent self-activation of innate immune response pathways²¹⁻²⁵. Considering this editing-mediated regulation of immunity, one proposed consequence of increased editing levels in cancer cells is repressed interferon production, leading to sustained cell growth²⁶. However, in certain cancer types, global editing levels were lower in tumors than in matched normal samples¹⁸, at least based on analysis of bulk tumors. As tumors are highly heterogeneous with respect to cancer cell genomics and tumor microenvironment^{27,28}, whether and how much editing aberration occurs in different subclones within tumors are important questions to address.

There is a need for new methods and materials for the analysis of RNA editing in cancer cells (e.g., lung cancer cells) that can identify RNA mutations that correlate with tumor mutation burden and cancer innate immunity. The present disclosure provides solutions to this need.

### SUMMARY OF THE INVENTION

As disclosed herein, we have characterized RNA editing in single cells of tumor and non-malignant biopsies collected at different treatment stages of lung cancer patients. Our analysis reveals that cancer cells are the main cell type that carries the elevated editing signature in bulk tumors. Building upon these discoveries, we then identified a significant correlation between tumor mutation burden (TMB) and RNA editing levels, a discovery supporting the notion that RNA editing creates RNA mutations, a previously under-appreciated source of cancer mutations. Specifically, we have discovered that the burden of RNA-level mutations, quantitated as the RNA editing load, negatively correlates with innate immune signatures in cancer cells and better predicts patient survival than TMB and innate immune profiles. These analyses present a global view of the RNA editing landscape in distinct cell types of lung cancer and the potential association of RNA editing with tumor immunity and patient survival.

As discussed below, the invention has a number of embodiments. Embodiments of the invention include, for example, methods of obtaining information useful for predicting patient survival in a patient diagnosed with a cancer (e.g., a lung cancer or a colorectal cancer). Typically these methods comprise obtaining a cancerous cell from the patient; obtaining double stranded RNA from the cancerous cell; observing double stranded RNA editing in the RNA obtained from the cancerous cell; and then correlating the double stranded RNA editing observed with patient survival such information useful for predicting patient survival in a patient diagnosed with a cancer is obtained. Typically, these methods also include: obtaining a noncancerous cell from the patient; obtaining double stranded RNA from the noncancerous cell; observing double stranded RNA editing in the RNA obtained from the noncancerous cell; and then comparing levels of double stranded RNA editing observed in the cancerous cell with levels of double stranded RNA editing observed in the noncancerous cell. In certain embodiments, the methods include observing amounts/load of edited dsRNA in the cell. For example, in certain embodiments of the invention, an RNA editing load observed in the cancerous cell that is at least 10%, 20% or 30% greater than the editing load observed in the noncancerous cell provides evidence of likely tumor progression and/or resistance to treatment with a chemotherapeutic agent. In certain embodiments of the invention, the double stranded RNA editing observed is correlated with patient response to a therapeutic intervention, tumor mutation burden or cancer innate immunity.

In certain embodiments of the invention, dsRNA editing is observed in one or more selected genes. For example, in some embodiments of the invention, RNA editing is observed in at least one double stranded RNA transcribed from a cathepsin B gene, a small integral membrane protein 14 gene, or an aryl hydrocarbon receptor gene. In some embodiments of the invention, double stranded RNA editing is observed in at least one double stranded RNA selected from: Alu-dsRNA: chr19:9635175-9635484 AluSp_SINE (these Alu locations/designations are those from human genome version HG-19); Alu-dsRNA: chr14:66737078-66737348 AluJb_SINE; Alu-dsRNA: chr21:34806547-34806850 AluSx1_SINE; and Alu-dsRNA: chr7:99960634-99060779 FLAM_C_SINE. Optionally, dsRNA editing observed is correlated with patient response to a therapeutic intervention, tumor mutation burden or cancer innate immunity. In embodiments of the invention, the method can include a multiplexed polymerase chain reaction process.

Related embodiments of the invention include methods of observing double stranded RNA (dsRNA) editing in a human cell comprising: obtaining dsRNA from one or more human cells (e.g., a single cell from a patient diagnosed with a skin cancer (e.g. melanoma), a lung cancer or a colorectal cancer); observing dsRNA editing in the dsRNA obtained from the cell, wherein dsRNA editing is observed in at least one of (1) dsRNA transcribed from a cathepsin B gene, a small integral membrane protein 14 gene, or an aryl hydrocarbon receptor gene; (2) dsRNA selected from: Alu-dsRNA: chr19:9635175-9635484 AluSp_SINE; Alu-dsRNA: chr14:66737078-66737348 AluJb_SINE; Alu-dsRNA: chr21:34806547-34806850 AluSx1_SINE; and Alu-dsRNA: chr7:99960634-99060779 FLAM_C_SINE; and/or (3) dsRNAs encoding a SRP9 polypeptide, a INADL polypeptide, a TAGLN polypeptide, a SLC35E3 polypeptide, a ARL13B polypeptide or a PARP8 polypeptide. In certain embodiments, the methods include observing amounts/load of edited dsRNA in the cell. Optionally, dsRNA editing observed is correlated with patient response to a therapeutic intervention, tumor mutation burden or cancer innate immunity. In embodiments of the invention, the method can include a multiplexed polymerase chain reaction process.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 Overview of editing events detected in single cells. A. Histogram of the number of single cells edited per editing site (x axis: log 10 scale). A site was considered edited in a cell if the site was covered by at least five reads and editing was supported by at least one read. B. Histogram of the number of sites edited per cell (x axis: log 10 scale). Editing criteria were the same as in A. C. Distribution of common editing sites in different types of regions. Common editing sites comprised sites edited in at least 50 cells. nc: non-coding. D. Distribution of common editing sites in Alu or non-Alu regions. E. Distributions of single cell editing levels at each of the two cancer-associated recoding sites across cell types. For the AZIN1 recoding site, p<0.05 (Mann Whitney U test) for pairwise comparisons of cancer vs the following cell types: T cells, macrophages, B cells, fibroblasts, mast cells. For the CCNI recoding site, p<0.001 (Mann Whitney U test) for pairwise comparisons of cancer vs the following cell types: pDCs, T cells, macrophages, B cells, epithelial cells, endothelial cells, fibroblasts, mast cells.

FIG. 2 Differential editing in distinct cell types of tumors. A. Counts of edited sites in single cells grouped by cell type, considering only the significantly tumor-increased editing sites from bulk tumors. A site was considered edited in a single cell if the site was covered by at least five reads and editing was supported by at least one read. p<=0.0001 when comparing cancer to any other cell type by Mann Whitney U test. B. Distribution of differences in mean editing levels between pooled cancer cells and pooled non-malignant epithelial cells. Only sites with significant differences (mean difference >=0.05, REDIT LLR adjusted p-value<0.05) are included. The number of significantly different editing sites is labeled on top. C. Similar to B but for differential editing sites between pooled cells of tumor samples and pooled cells of non-malignant samples for each non-epithelial cell type. D. Top gene ontology (GO) enrichment in genes containing differential editing sites between cancer cells and non-cancerous epithelial cells, compared to background genes without differential sites but matched according to gene length and expression. Only the 20 most significantly enriched terms, each with a minimum of 10 corresponding genes, are shown. E. Top GO enrichment among differentially edited genes in non-epithelial cell types found in tumors vs non-malignant samples. As in D, only the top 20 terms are included for each cell type, and background genes were chosen in the same manner. Larger circle size represents higher statistical significance of enrichment. Color of circle fill corresponds to cell type. Terms are grouped by broader categories labeled on the right.

FIG. 3 RNA editing differences across treatment time points. A. Overall editing levels of cancer cells grouped by treatment point. Overall editing level was calculated as the mean over editing levels of all sites for each cell. Editing levels were compared among treatment points by Mann Whitney U test, with p value significance shown. *p<=0.05. ***p<=0.001. ****p<=0.0001. B. Similar to A, but samples were further grouped by oncogenic driver mutations. C. Mean editing levels of differentially edited sites between paired treatment time points. In each scatterplot, the numbers of differential sites hyperedited and hypoedited are listed in the top left and bottom right corners, respectively. Deviation of the proportion of hyperedited sites from 0.5 was assessed by binomial test, with p value shown below the number of hyperedited sites. D. Proportions of sites differentially edited between PD and RD treatment time points among sites with (Drug-associated, blue) or without (None, red) associations to drug response. We considered only the reported editing-drug associations in TCGA LUAD for the same targeted therapies given to the lung scRNA-seq patients, most of which were associations with drug sensitivity.

FIG. 4 Editing in cancer cells associated with immune suppression. A. Hexagonal 2-dimensional histograms of mean editing level and mean normalized ISG expression in single cancer cells for multiple ISG signatures. Mean editing level was calculated using editing levels of all sites for each cell. Red asterisk indicates a significant negative spearman correlation, with FDR-adjusted p-value<0.05. B. Spearman correlations between mean editing levels and mean expression of multiple ISG signatures across single cancer cells, grouped by treatment time point. The number of cancer cells within each category is listed in the x-axis labels. The size of each circle indicates the magnitude of the Spearman correlation coefficient, and the color intensity corresponds to significance of the adjusted p-value. Blue: positive correlations, red: negative correlations. TN stands for treatment naïve. RD stands for residual disease, and PD stands for progression. C. Top: Bar plot showing Spearman correlations between cancer editing levels and infiltration of different immune cell types. For each tumor, single cancer cells were pooled, and overall cancer editing level was calculated as the mean editing level of all sites in the pooled cancer cells. Red bar: significant correlation with p<0.05. Nonsignificant correlations are shown in white. For each cell type, in parentheses, lung signifies that only lung biopsies were included. In contrast, “all” signifies all samples were included. Bottom: scatterplot of cancer editing and infiltration of Natural Killer cells, with Spearman correlation coefficient and p-value listed. D. Top: Bar plot of Spearman correlations between tumor-increased editing and estimated infiltration of different immune cell types in bulk TCGA LUAD tumors. Tumor-increased editing was calculated as the mean editing level over sites with significantly higher editing levels in tumors than in matched non-malignant samples. Colored bars indicate significance by FDR-adjusted p<0.05, with positive correlations in blue and negative ones in red. Nonsignificant correlations are shown in white. Bottom: scatterplot of tumor-increased editing and quanTIseq-estimated proportion of Natural Killer cells with Spearman correlation coefficient and p-value shown.

FIG. 5 Relationship between RNA editing and tumor mutation burden specific to cancer cells. A. Left bar plot shows statistical significance (log 10-transformed adjusted p value) of estimated TMB in multiple linear models predicting mean editing levels of each cell type. Total read coverage was included as a covariate in the linear models. Blue fill color indicates significance by FDR 5% (sig), and associations that were not significant are shown as white bars (ns). Scatterplot shows positive association between TMB and RNA editing load in cancer cells, with Spearman correlation coefficient and corresponding p value labeled. B. Scatterplot of TMB and RNA editing load in bulk TCGA LUAD tumors, labeled with Spearman correlation coefficient and p value. On the right, distributions of TMB across tumors grouped by RNA editing load tertiles. C. Scatterplot of Pearson correlation coefficient and statistical significance of associations between gene editing levels and TMB in TCGA LUAD. Purple indicates significance by passing FDR 10% and minimum correlation coefficient of 0.15 (sig). Nonsignificant correlations are shown in gray (ns). D. Gene ontology terms enriched among genes with editing levels significantly correlated with TMB (from C), compared to background non-correlated genes with similar gene length and expression levels. Bar length represents statistical significance of enrichment, and vertical gray line indicates a threshold of FDR 10%. E. Forest plot showing hazard ratio estimates and p-values obtained by fitting a Cox regression model with TMB, editing load (as a percent value), age, and gender as covariates. F. Performance of Cox Proportional Hazards models on editing load, TMB, and ISG expression individually or jointly to predict overall survival in TCGA LUAD patients. Distribution of c-index from 5-fold cross-validation is shown for each model. G. GO biological process terms most strongly enriched in genes differentially expressed between high and low editing groups. TCGA LUAD tumors were grouped by editing load tertiles, as in B. Enrichment was tested separately for genes upregulated in the high editing group (up, teal) and genes downregulated in the high editing group (down, orange). Only the top 30 terms enriched in each direction are included. Larger point size indicates higher statistical significance of enrichment. Broader categories for terms are indicated by bar color on the right.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention.

As discussed below, an analysis of RNA editing in cancer cells identified RNA mutations in such cells that correlate with tumor mutation burden and cancer innate immunity. In addition, the amount of RNA mutations strongly predicts patient survival. Building upon these discoveries, we have developed methods and materials useful for the single cell dissection of RNA editing in cancer and then correlating the significance of the observed RNA editing load with the patient's cancer prognosis. Methods and materials pertaining to RNA editing that can be adapted to embodiments of the invention are disclosed, for example, in US Patent Application Publication numbers: 20210172961 and 20170191057; Chan et al., Genome Biol. 2020 Oct. 26; 21(1):268. doi: 10.1186/s13059-020-02171-4; and Kung et al., Front Endocrinol. 2018 Dec. 18; 9:762. doi: 10.3389/fendo.2018.00762, the contents of which are incorporated by reference.

In an illustrative working embodiment of the invention, we analyzed lung adenocarcinoma (LUAD) single cell RNA-seq data and bulk tumor RNA-seq data. We discovered that a tumor-specific RNA editing load predicts patient survival much more effectively than tumor mutation burden (TMB). RNA editing load is defined as the average RNA editing level of tumor-specific RNA editing sites. This metric can be calculated by collecting RNA samples from a tumor biopsy, and measure RNA editing level in a high-throughput manner.

As discussed below, we have developed a technology to measure RNA editing load that involves multiplexed PCR and sequencing. Based on bioinformatic analysis of tumor RNA-seq data (such as for LUAD), we first identify a list of tumor-specific RNA editing sites. For these sites, we design specific multiplexing PCR primers to amplify them, the product of which constitutes a library for high throughput sequencing. This cost-effective way of profiling multiple RNA editing sites per tumor provides an accurate measure of RNA editing load. In this context, double-stranded RNAs (dsRNAs) are potent activators of immune response. If edited via the RNA editing machinery, their potency for immune activation is reduced. Thus. RNA editing is a mechanism to modulate dsRNA immunogenicity.

In one illustrative working embodiment of the invention, an analysis of LUAD RNA-seq data revealed a number of dsRNAs (e.g., those in the genes CTSB, SMIM14, AHR) including Alu-dsRNAs whose editing levels were associated with response to immunotherapy and patient survival. For example, identified a specific dsRNA in the gene CTSB that had the most potent predictive power of patient survival after immunotherapy. This is the same dsRNA discovered in the lung cancer study. Specifically, patients with an unedited CTSB dsRNA expression level >33 had better survival those patients with lower expression following immunotherapy. Moreover, in our analysis of melanoma RNA-seq data, we discovered that a higher number of unedited dsRNA in a tumor sample predicts better patient survival after immunotherapy. The unedited dsRNA was typically defined as those with unedited dsRNA expression level >20.

As discussed below, the invention has a number of embodiments. Embodiments of the invention include, for example, methods of obtaining information useful for predicting patient survival in a patient diagnosed with a cancer (e.g., a lung cancer or a colorectal cancer). Typically these methods comprise obtaining a cancerous cell from the patient; obtaining double stranded RNA from the cancerous cell; observing double stranded RNA editing in the RNA obtained from the cancerous cell; and then correlating the double stranded RNA editing observed with patient survival such information useful for predicting patient survival in a patient diagnosed with a cancer is obtained. Typically, these methods also include: obtaining a noncancerous cell from the patient; obtaining double stranded RNA from the noncancerous cell; observing double stranded RNA editing in the RNA obtained from the noncancerous cell; and then comparing levels of double stranded RNA editing observed in the cancerous cell with levels of double stranded RNA editing observed in the noncancerous cell. In certain embodiments, the methods include observing amounts/load of edited dsRNA in the cell. For example, in certain embodiments of the invention, an RNA editing load observed in the cancerous cell that is at least 10%, 20% or 30% greater than the editing load observed in the noncancerous cell provides evidence of likely tumor progression and/or resistance to treatment with a chemotherapeutic agent. In certain embodiments of the invention, the double stranded RNA editing observed is correlated with patient response to a therapeutic intervention, tumor mutation burden or cancer innate immunity.

In certain embodiments of the invention, RNA editing is observed in at least one double stranded RNA transcribed from a cathepsin B gene (Cathepsin B (CTSB); Gene ID: 1508, Chromosomal Location: 8p23.1), a small integral membrane protein 14 gene (Small integral membrane protein 14 (SMIM14); Gene ID: 201895, Chromosomal Location: 4p14), or an aryl hydrocarbon receptor gene (Aryl hydrocarbon receptor (AHR); Gene ID: 196 Chromosomal Location: 7p21.1). In some embodiments of the invention, double stranded RNA editing is observed in at least one double stranded RNA selected from: Alu-dsRNA: chr19:9635175-9635484 AluSp_SINE; Alu-dsRNA: chr14:66737078-66737348 AluJb_SINE; Alu-dsRNA: chr21:34806547-34806850 AluSx1_SINE; and Alu-dsRNA: chr7:99960634-99060779 FLAM_C_SINE. Optionally, dsRNA editing observed is correlated with patient response to therapeutic intervention, tumor mutation burden or cancer innate immunity. In embodiments of the invention, the method can include a multiplexed polymerase chain reaction process.

Related embodiments of the invention include methods of observing double stranded RNA (dsRNA) editing in a human cell comprising: obtaining dsRNA from one or more human cells (e.g., a single cell from a patient diagnosed with a skin cancer, a lung cancer or a colorectal cancer); observing dsRNA editing in the dsRNA obtained from the cell, wherein dsRNA editing is observed in at least one of: (1) dsRNA transcribed from a cathepsin B gene, a small integral membrane protein 14 gene, or an aryl hydrocarbon receptor gene; (2) dsRNA selected from: Alu-dsRNA: chr19:9635175-9635484 AluSp_SINE; Alu-dsRNA: chr14:66737078-66737348 AluJb_SINE; Alu-dsRNA: chr21:34806547-34806850 AluSx1_SINE; and Alu-dsRNA: chr7:99960634-99060779 FLAM_C_SINE; and/or (3) dsRNAs encoding a SRP9 polypeptide, a INADL polypeptide, a TAGLN polypeptide, a SLC35E3 polypeptide, a ARL13B polypeptide or a PARP8 polypeptide. In certain embodiments, the methods include observing amounts/load of edited dsRNA in the cell. Optionally, dsRNA editing observed is correlated with patient response to a therapeutic intervention, tumor mutation burden or cancer innate immunity. In embodiments of the invention, the method can include a multiplexed polymerase chain reaction process. In certain embodiments of the invention, the dsRNA editing observed results in at least one (two, three or four) alterations in a polypeptide sequence selected from: Q49R in the human signal recognition particle 9 (“SRP9”: Gene ID: 6726) polypeptide; R1146G in the human InaD-like (Drosophila) (INADL), mRNA (“INADL”: NM_176877) polypeptide; D23G in the human transgelin (“TAGLN”: Gene ID: 6876) polypeptide; S56G in the human Solute Carrier Family 35 Member E3 (“SLC35E3”: Gene ID: 55508) polypeptide; S61G in the human ADP-ribosylation factor-like protein 13B (“ARL13B”: Gene ID: 200894) polypeptide; and/or I97M in the human poly(ADP-ribose) polymerase family member 8 (“PARP8”: Gene ID: 79668) polypeptide.

Various illustrative aspects and embodiments of the invention are discussed in Chan et al., Cancer Res. 2023 Feb. 3:83(3):374-385 (hereinafter “Chan et al.”) which is incorporated herein by reference, and the following sections.

### Materials and Methods

### Single Cell RNA-Seq Data Processing and Cell Type Assignment

We downloaded scRNA-seq fastq files from BioProject PRJNA591860²⁹. After checking data quality with FastQC version 0.11.5 (median base sequence quality ≥25, lower quartile for base sequence quality >10, and no adapter sequences present in over 5% of reads) and aligning reads to the human genome with STAR³⁰ (version 2.7.0e), we ran HTSeq (version 0.12.4) count to obtain gene-level abundance with --mode=intersection-nonempty. Using the R package Seurat³¹ (version 4.0.0), we combined these count data into a Seurat object and retained cells based on the following quality control metrics: sequencing depth (>50,000 reads), features (>500 genes), and mitochondrial read content (<50%). For these remaining cells, abundance profiles were normalized by sctransform³² (version 0.3.2) before linear dimensional reduction with principal components analysis (PCA). We used the top 20 PCs, chosen based on an elbow plot, to cluster cells (with cluster resolution=0.09) and run non-linear dimensional reduction by UMAP. Based on abundance of broad compartment marker genes for epithelial (EPCAM), endothelial (CLDN5), immune (PTPRC), and stromal (COL1A2) cells³³, we labeled clusters initially as immune and non-immune cells.

Next, we subclustered non-immune cells after running sctransform and PCA on only non-immune cells (20 PCs, cluster resolution=0.12). Each cluster was assigned a cell type based on its abundance of known cell type marker genes²⁹. Alveolar cells were grouped with epithelial cells for downstream analyses. To distinguish cancer cells from non-malignant epithelial cells, we applied inferCNV³⁴ to estimate chromosome- or arm-size CNVs in epithelial cells with fibroblasts and endothelial cells as reference and spike-in controls. Specifically, amplification and deletion regions were approximated through comparing RNA abundance profiles of input cells to those of labeled reference cells (1000 fibroblasts and 500 endothelial cells). Input cells included all epithelial cells, as well as the spike-ins: 1000 fibroblasts and 500 endothelial cells.

Immune cells were subclustered with the same procedure, using 24 PCs and cluster resolution=0.07. Including only lung biopsies, we also further subclustered T cells (20 PCs, resolution=0.18) and macrophages (11 PCs and resolution=0.2) separately in the same manner.

### Bulk RNA-Seq Data Processing

We downloaded RNA-seq fastq files for tumor and non-malignant samples of the TCGA Lung Adenocarcinoma (LUAD) project from the Genomic Data Commons (GDC) Legacy Archive³⁵. Reads were first mapped with HISAT2³⁶ (version 2.0.5) using the following parameters: q, phred33, no-softclip, add-chrname, no-unal, reorder, no-discordant, no-mixed, un-conc-gz. To account for hyperedited regions that could cause many mismatches to the reference genome in reads, we ran a hyperediting pipeline^{37,38} on initially unmapped reads. Reads rescued from this pipeline were combined with uniquely mapped reads from the first round of alignment.

### Identification of RNA Editing Sites

Using our previously published methods^{10,37-39,40}, we detected editing events within each single cell at sites recorded in the REDIportal v2⁴¹ database. If an editing site overlapped a variant listed in dbSNP (version 147) or COSMIC (version 81) and was not previously reported as a cancer-associated editing site^{18,26,42-48} we excluded the site from downstream analyses.

Similarly, we quantified editing levels at REDIportal sites in bulk tumor and matched non-malignant tissue samples from TCGA⁴⁹ LUAD. In addition to removing sites within dbSNP and COSMIC databases, we filtered out editing events overlapping sample-specific somatic mutations and copy number alteration data available at the TCGA.

### Differential Editing by Cell Type

To compare editing levels between tumor and non-malignant samples in a single cell type, we first pooled counts of edited and unedited reads of single cells of that cell type within each sample. We used only tumor samples from the three patients with matched non-malignant samples. Treating pooled cells of each sample as a replicate (5 tumors and 3 non-malignant controls) for each cell type, we ran REDIT-LLR⁵⁰ to test for the editing difference between tumors and controls. A site was considered testable if it was covered by at least 5 reads in at least one pooled sample in each condition. Significance of editing differences was determined by REDIT FDR-adjusted p-value<0.05 and difference in mean editing levels ≥0.05.

### Differential Editing in Bulk Tumors

We used REDIT-regression⁵⁰ to identify sites that were differentially edited between bulk tumors and non-malignant samples from TCGA LUAD. For each testable editing site, the following covariates were considered in the regression model: gender, race, age, and sample type (tumor or non-malignant). An editing site was defined as differential if the sample type FDR-adjusted p-value<0.05 and the difference in mean editing levels ≥0.05.

### Calculation of RNA Editing Load

For each tumor in the scRNA-seq data, cancer cells were pooled, and mean editing level was calculated over all sites in the pooled cells. RNA editing load was defined as this mean editing level in cancer cells per tumor. Mean editing level was calculated in the same way for other cell types.

To define the RNA editing load for each bulk tumor, we focused on the differentially edited sites identified using REDIT-regression as described above. Among the differential sites, we obtained the subset that had significantly higher editing levels in tumors than in non-malignant samples and named them tumor-increased sites. We then calculated the mean editing level of these tumor-increased sites for each tumor and defined this value as the RNA editing load of each tumor.

### Quantification of Tumor Mutation Burden

We downloaded the list of prioritized somatic point mutations provided for individual cancer cells (Table S3 of ref²⁹). For each tumor sample, tumor mutation burden was estimated as the number of unique nonsynonymous mutations from this list across cancer cells in that sample. This mutation list was also used to filter editing events in cancer cells before correlating TMB and editing levels.

For each bulk TCGA LUAD tumor, we calculated tumor mutation burden as the number of unique somatic nonsynonymous, stop-gain or stop-loss point mutations.

### Immune Cell Infiltration Estimates

We downloaded quanTIseq⁵¹ estimates of immune cell proportions in TCGA tumors from TIMER2.0 (http://timer.cistrome.org)⁵². The quanTIseq method was chosen due to the interpretability of its output as cell fractions and the inclusion of more immune subtypes.

### Survival Prediction by RNA Editing and Other Molecular Markers

To fit a Cox proportional hazards regression model with RNA editing load, TMB, age, and gender, we used the coxph function of the R package survival. From this same package, we used the cox.zph function to test the proportional hazards assumption.

Using the Python module scikit-survival, we fit a Cox proportional hazards model to each of the following metrics individually for overall survival in TCGA LUAD patients: RNA editing load (as a percent value), TMB, and mean RNA abundance of each of six ISG sets (IFNalpha. IFNgamma, ISGdsrna, ISGliu, ISGchronic, ISG RS). We also fit six additional Cox models to combinations of features, specifically a model for each ISG signature plus RNA editing load and TMB. Performance of these survival prediction models was assessed with c-index (Harrell's concordance index) from five-fold cross-validation.

### Results

### Characterization of RNA Editing in Single Cells

To examine RNA editing profiles of different cell types in lung cancer, we analyzed single-cell (sc) RNA-seq data (produced via Smart-seq2) from tumors (n=46) and tumor adjacent tissues (TATs, n=3) of 30 non-small cell lung cancer (NSCLC) patients²⁹. After filtering by quality control metrics, we clustered cells in multiple rounds based on normalized RNA abundance profiles and assigned cell type labels using marker genes (FIG. S1 in Chan et al., Methods). Briefly, we labeled clusters initially as immune or non-immune, then subclustered these broad types separately. We annotated immune subtypes as B cells, T cells, mast cells, macrophages, neutrophils, and plasmacytoid dendritic cells (pDCs). From non-immune clusters, we obtained epithelial cells, endothelial cells, fibroblasts, hepatocytes, and melanocytes. Cancer cells were distinguished from non-malignant epithelial cells by comparing somatic copy-number aberration (CNA) estimates to those of reference fibroblasts and endothelial cells³⁴ (FIG. S2A-B in Chan et al.). After removing PCR duplicates, single cells had 0.466 million uniquely mapped reads on average (FIG. S2C in Chan et al.).

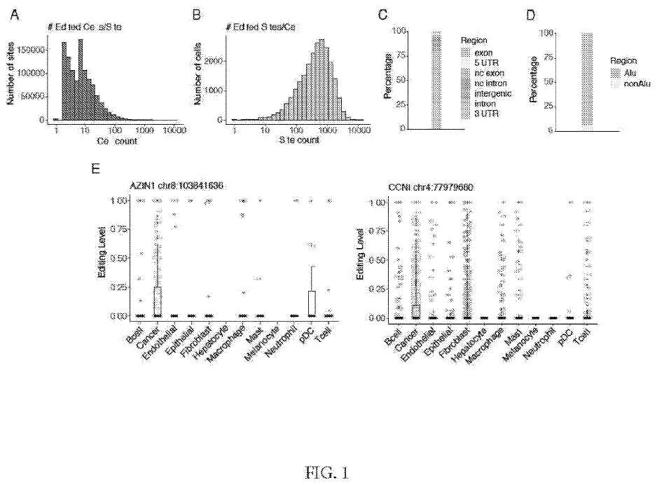

We identified editing sites in each cell using previously published methods^{37,39,40} and requiring their presence in the REDIportal v2 database⁴¹. On average, an individual editing site was found to be edited (with ≥1 edited read and ≥5 reads in total coverage) in 14 cells and covered (with ≥5 total reads) in 314 cells (FIG. 1A, FIG. S3A in Chan et al.). In an individual cell, a mean of 672 sites were edited and 15,037 were covered (FIG. 1B, FIG. S3B in Chan et al.). Of the 1,096,361 sites edited in one or more cells, 50,576 sites were edited in at least 50 cells. These were located primarily in introns and 3′UTRs, within Alu regions (FIG. 1C-D). To estimate the general editing level of a single cell, we calculated the mean of editing levels of all sites in the cell. Consistent with ADAR1 expression levels, overall editing levels were highest in cancer cells (FIG. S3C-D in Chan et al.).

Next, we examined the editing levels of two recoding sites (in AZIN1 and CCNI respectively) known to have higher editing levels in adenocarcinomas of the lung than in non-malignant lung tissue⁵³. Both sites have been shown to have cancer-specific functions^{2,6-8}. Consistent with these reported findings, we observed the highest recoding rates in cancer cells at both sites (FIG. 1E). These results support the validity of the single cell RNA editing profiles derived here.

### Elevated Editing Observed in Cancer Cells, but not Other Cell Types

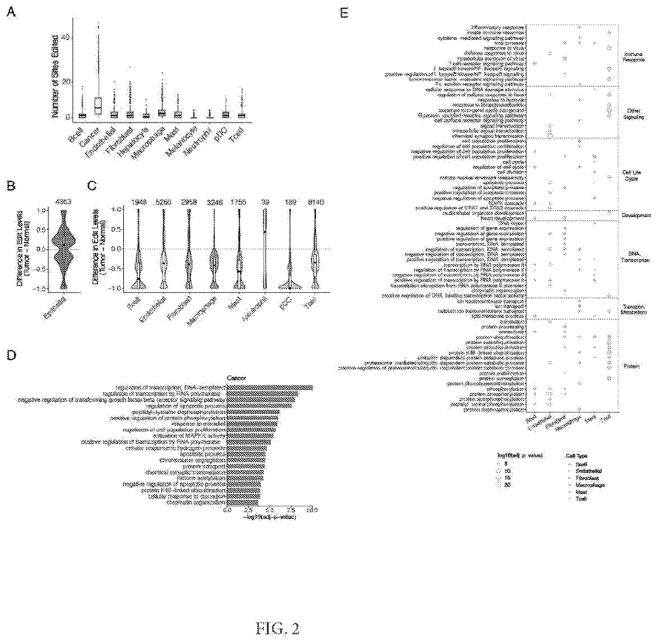

Previous studies detected numerous editing changes in bulk RNA-sequencing of lung adenocarcinomas compared to matched tumor-adjacent normal tissue^{18,19}, a finding we reproduced by analyzing tumour and non-malignant tissue samples in the TCGA project (FIG. S4 in Chan et al., Methods). There exists a global trend of higher editing levels in LUAD tumors relative to matched non-tumor lung tissue. Considering the heterogeneity of the tumor microenvironment observed across patients^{54,55}, we asked whether tumor-associated editing aberrations were cell-type-specific. To address this question, we first obtained the editing frequencies of tumor-increased sites in the bulk LUAD data (i.e., differential sites in FIG. S4 in Chan et al. that had higher editing levels in the tumors), and examined their editing levels across cell types in the scRNA-seq data. These sites were most frequently edited in cancer cells compared to other cell types (FIG. 2A). Consistent with ADAR1 overexpression in cancer cells (FIG. S3D in Chan et al.), editing levels were most strongly correlated with ADAR1 expression in cancer cells, compared to other cell types (FIG. S3E in Chan et al.). These observations suggest that heightened editing in bulk tumors likely reflect altered editing in cancer cells more often than in other cell types present in tumors.

To further investigate the specificity of tumor-associated editing in individual cell types, we applied REDIT LLR⁵⁰ to test for editing differences for each cell type found in tumors vs. non-malignant controls in the scRNA-seq data. Using the eight samples from the three patients with TATs, we pooled cells of the same cell type within a sample. For non-epithelial cell types, we compared editing levels of pooled cells in tumor samples to those in non-malignant tissues. Considering cancer cells as the tumor condition of epithelial cells in LUAD, we compared editing levels of pooled cancer cells to pooled non-malignant epithelial cells. Strikingly, cancer cells alone displayed a dominant trend of increased editing compared to non-malignant epithelial cells (FIG. 2B). In contrast, editing levels were generally reduced in the other cell types found in tumors compared to the same respective cell types found in non-malignant tissues (FIG. 2C). It should be noted that the general trends in overall editing and differential editing remain the same given variable coverage requirements (minimum number of edited reads or total reads, FIG. S5 in Chan et al.).

Six recoding sites were more highly edited in cancer cells compared to non-malignant epithelial cells (Table S1 in Chan et al.). Notably, one of these sites is in TAGLN, which was shown to enhance cancer cell invasiveness in NSCLC and colorectal cancer⁵⁶⁻⁵⁷. Furthermore, higher recoding levels in INADL and ARL13B have the potential to impact cancer cell migration as well, since these genes have reported roles in tight junction and cilia formation^{58,59}.

We next sought to determine whether the types of genes harboring tumor-associated editing differ by cell type. To this end, we performed a gene ontology (GO) enrichment analysis on differentially edited genes in each cell type. Enrichment was assessed against background genes with gene length and RNA abundances comparable to differentially edited genes, as described previously^{10,37}. While several categories, such as cell proliferation and apoptotic process, were identified across multiple cell types, certain pathways appeared to be enriched in only specific cell types (FIG. 2D-E). For instance, enrichment of regulation of transforming growth factor beta receptor signaling pathway and cytoskeleton organization were exclusive to cancer cells. Moreover, the inflammatory pathways of tumor necrosis factor-mediated signaling pathway and interleukin-1-mediated signaling pathway were significantly enriched in differential editing of T cells only. Considering these findings together, tumor-associated editing changes appear distinct across cell types and occur in cancer-relevant pathways.

### Differential Editing in Cancer Cells Across Treatment Time Points

The above analysis examined all tumor samples regardless of their stage, treatment status or driver mutations. The results supported existence of an overall trend of changes of RNA editing in different cell types of LUAD. In this particular dataset, clinically actionable driver mutations had been reported, and samples were acquired from patients at one of three treatment time points or statuses: 1) before systemic targeted therapy, i.e., treatment naïve (TN), 2) residual disease (RD, where the tumor was regressing or stable by clinical imaging), and 3) progressive disease (PD, where the tumor showed acquired drug resistance) (Table S2 in Chan et al.). Since targeted therapies were developed based on cancer cell mutations, we focused on cancer cells in the analyses below.

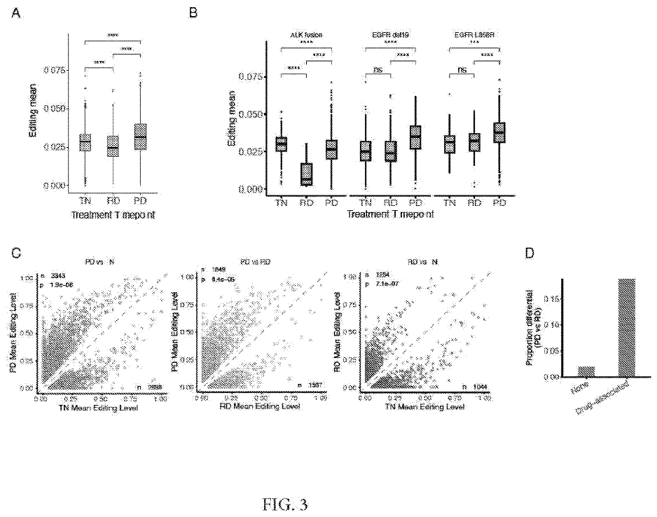

Interestingly, we observed significant differences in overall editing levels in cancer cells across treatment time points (FIG. 3A), where the PD samples had the highest editing levels, followed by the TN samples. In contrast, the RD group showed the lowest overall editing level. Since RD samples represent regressing tumors, this observation supports that cancer cells in regressing tumors had lower editing, indicating their resemblance of normal cells in terms of editing profiles. In addition, the higher editing in PD samples suggests that increased editing is likely a signature of progressive tumors with drug resistance.

For three driver mutations (ALK fusion, EGFR del19 and EGFR L858R), samples from all three treatment points were collected (Table S2 in Chan et al.). We thus compared the overall editing levels across the three points for each driver mutation. Consistent with the above results, the PD group had the highest editing among samples with EGFR mutations, whereas the RD group showed lowest editing among samples with ALK fusion (FIG. 3B). Note that ADAR expression across treatment points was not well correlated with editing levels, suggesting the possible presence of other regulatory mechanisms (FIG. S6A-B in Chan et al.).

As overall editing levels differed across treatment time points, we next assessed differences in editing levels at individual sites between pairs of treatment points. A total of 6,241, 3,436 and 2,328 sites were identified as differential for PD vs. TN, PD vs. RD, and RD vs. TN, respectively (FIG. 3C). Consistent with the above findings, more differential editing sites were observed to be higher edited in the PD group than the other two groups. Genes differentially edited in the PD status included those related to immune regulation, drug resistance and EMT processes (FIG. S6C in Chan et al.), indicating the potential relevance of RNA editing to tumor progression reflected in PD.

Since PD samples possess drug resistance to targeted therapy, we next examined the overlap between differentially edited sites and editing sites correlated with drug response from the GPEdit database⁶⁰. For this analysis, we focused on editing differences between PD and RD because both groups underwent targeted therapy but demonstrated different responses. We observed that drug-associated editing events (in particular, drug sensitivity-associated) are more frequently differentially edited between PD and RD statuses compared to editing sites unassociated with drug response (FIG. 3D; Pearson's Chi-squared p-value=0.0005). Potentially contributing to multiple hallmarks of cancer, genes with such editing sites have roles in cell polarity (PATJ), cell cycle (MDM2), tumor suppression (METTL7A), and innate immunity (MAVS). These drug response associations support the potential relevance of RNA editing to targeted therapy resistance.

Cancer Editing Associated with Immune Suppression

As a contributor to innate immune suppression, ADAR marks endogenous dsRNAs by RNA editing so that these dsRNAs do not unnecessarily activate cytosolic sensors, such as MDA5 and PKR, and their signaling pathways, which would lead to IFN production, translational repression, or growth arrest^{24,61,62} Consistent with this role of ADAR in normal tissues, certain cancer cells were found to be vulnerable to ADAR loss through stimulation of ISGs and growth inhibition in both in vitro and in vivo models^{14,16,17}. Cancer cells may upregulate RNA editing to exploit this mechanism of innate immune suppression.

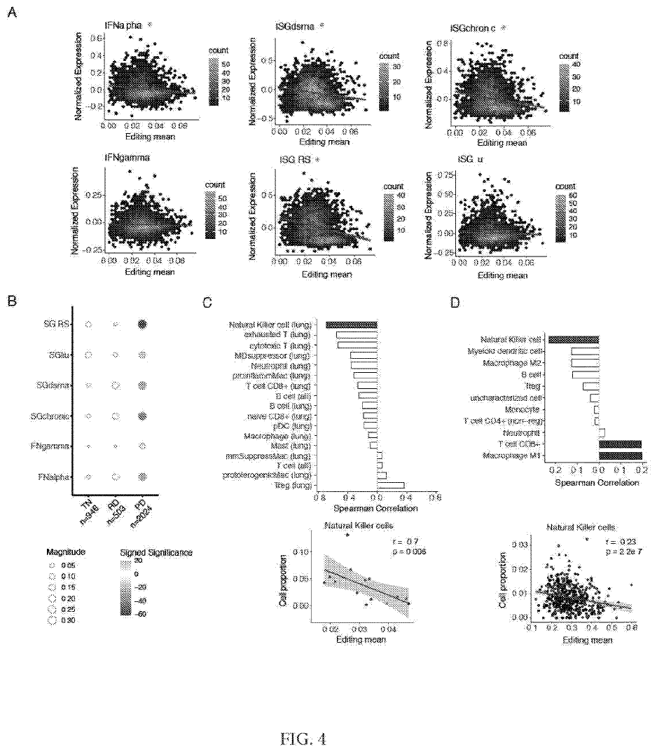

To explore this model in human tumors, we examined the correlation between RNA editing and ISG expression in cancer cells. For multiple sets of ISGs, we quantified their overall RNA abundance as the mean RNA abundance of genes in the set. The following sets of ISGs were included, the Hallmark IFN-alpha and IFN-gamma response gene sets, 60 genes suppressed by IU-dsRNA during poly(IC) transfection⁶³ (ISGdsRNA), 38 cancer-specific genes associated with resistance to ICB⁶⁴ (ISG RS), 38 genes associated with ADAR dependence¹⁷ (ISGliu), and 30 genes with prolonged IFN-induced expression^{65,66} (ISGchronic). We observed that overall editing levels were negatively associated with ISG RNA abundance across single cancer cells for several ISG sets (FIG. 4A), consistent with the model of editing-mediated suppression of IFNs in cancer cells. Furthermore, grouping cancer cells by treatment timepoint, we observed most evident negative associations in the PD state (FIG. 4B).

As ISGs regulate activation and recruitment of different immune cells⁶⁷, Ne asked whether editing levels in cancer cells are also linked to the infiltration of immune cell types in tumors. For this analysis, we subclustered T cells and macrophages of lung tumors separately (FIG. S7A-B in Chan et al.) and calculated the proportions of cell types within each tumor, using our annotations of single cells. To measure a cancer-specific editing index in each tumor, we calculated the mean of editing levels across pooled cancer cells in the tumor. While a negative association between cancer editing and cell proportion was statistically significant only for natural killer (NK) cells, most of the other immune subpopulations also exhibited the same trend (FIG. 4C). In addition, cancer cell editing remained negatively correlated with NK cell proportion while controlling for cancer cell ISG signaling (FIG. S8A in Chan et al.). Thus, reduced immune infiltration is associated with higher editing levels in cancer cells, which may be only partly explained by the above observed suppressed interferon signaling in higher edited cancer cells (FIG. 4A-B).

In bulk lung adenocarcinomas from TCGA, we also observed a significant negative correlation between mean editing levels over tumor-increased sites and NK infiltration, as estimated by quanTIseq⁵¹ (FIG. 4D). In contrast, tumor-increased editing levels were positively associated with proportions of CD8+ T cells and M1 macrophages. In agreement with this observation, overall editing levels were highest in tumors of the C2 immune subtype (FIG. S8B in Chan et al.), characterized by the highest M1 and CD8+ T cell signatures among all immune subtypes identified across TCGA cancer types⁶⁸.

### Correlation Between RNA Editing Load and Tumor Mutation Burden

The above results support the relationship between RNA editing and cancer immunity. Another factor linked to cancer immunity is tumor mutation burden (TMB)⁶⁹⁷¹. Thus, we next examined the relationship between RNA editing and TMB, which is of particular interest because RNA editing effectively creates RNA mutations, analogous to DNA mutations expressed in the RNA. Thus, we quantified overall editing level as the RNA editing load, defined simply as the mean editing level of cancer cells in scRNA-seq of a tumor (Methods). In the bulk RNA-seq data, RNA editing load is calculated as the mean tumor-increased editing level per tumor.

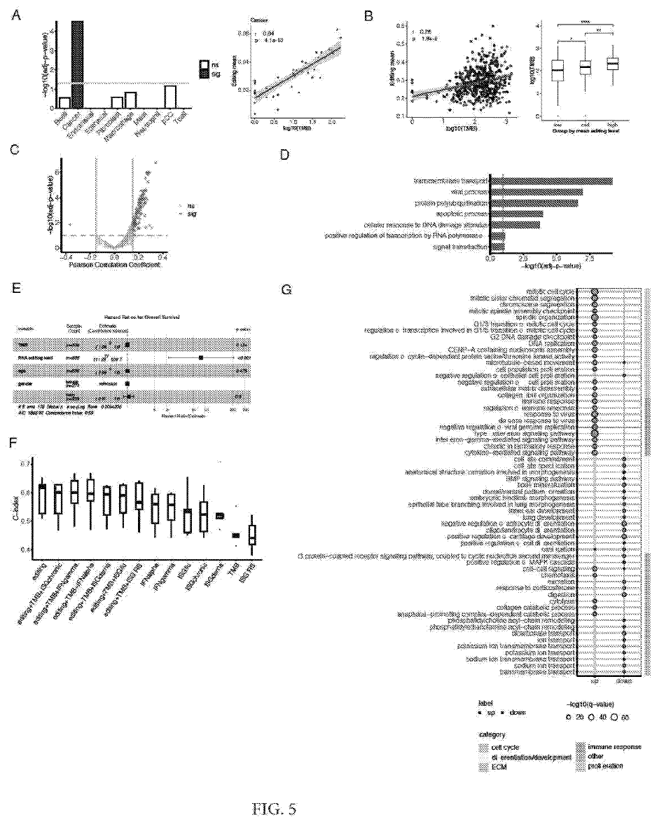

Using these definitions, we analyzed the correlation between RNA editing load and TMB in cancer cells identified in the scRNA-seq data. A strong positive correlation was observed between these two metrics (FIG. 5A). Note that no correlation was observed between the analogous RNA editing load calculated in non-cancerous cells and TMB. Assessing this apparent relationship in bulk tumors, we correlated RNA editing load (using tumor-increased editing sites in FIG. S4 in Chan et al.) and TMB in the LUAD data from TCGA. Consistent with the observation in the scRNA-seq data, higher RNA editing load corresponded to higher mutation burden (FIG. 5B).

To further examine the relationship between editing and DNA mutations, we tested the correlations between editing of individual genes and overall TMB in bulk tumors. Strikingly, nearly all significant associations between gene-specific editing load and TMB were positive (FIG. 5C). Additionally, significantly correlated genes were enriched for cancer-relevant GO categories such as apoptotic process and cellular response to DNA damage stimulus (FIG. 5D). The latter may contribute to the load of DNA mutations in tumors.

### High Editing Levels Mark Worse Patient Survival

We next considered the prognostic values of both RNA editing and TMB on the survival of lung adenocarcinoma patients. To do so, we fit a Cox proportional hazards model with TMB, RNA editing load, age, and sex. Of all the covariates included in the model, only RNA editing load was significantly associated with overall survival (FIG. 5E), suggesting that increased RNA editing is an important factor in LUAD prognosis.

Given the apparent correlations between RNA editing load, TMB and ISG signatures described above, we asked which molecular feature(s) were most relevant to overall survival. To compare their individual and collective predictive power, we fit Cox proportional hazards models to these features separately and to combinations of features in the TCGA data. To quantify IFN response signatures, we used the mean abundance of each of the same six ISG sets considered earlier. Across all individual features, RNA editing load was the most predictive of patient survival (FIG. 5F). Furthermore, performance of feature combinations was largely similar to that of editing alone, emphasizing the prominent role of editing load in these survival models.

We then tested whether RNA editing load offers additional prognostic value beyond cancer stage, which highly impacts prognosis and treatment decision-making. By fitting a Cox proportional hazards model with RNA editing load and stage, we found that RNA editing load remains significantly associated with survival while controlling for stage (FIG. S9 in Chan et al.).

To further understand the implications of RNA editing in patient survival, we investigated differential gene expression depending on editing load. After grouping bulk LUAD tumors according to tertiles of RNA editing load, we compared RNA abundance between high and low editing groups. Among genes with significantly increased expression in the high editing group, many cell cycle categories were enriched (FIG. 5G). Altered cell cycle regulation allows cancer cells to continue dividing, bypassing DNA damage and other checkpoints and avoiding potential apoptosis^{72,73}. Concomitantly, DNA damage may accumulate, increasing TMB. Also upregulated were genes involved in extracellular matrix disassembly, which is characteristic of the mesenchymal cell phenotype, following epithelial-mesenchymal transition (EMT). Along with this mesenchymal feature, significantly reduced genes in highly edited tumors were overly represented with GO categories related to cell fate and differentiation. This downregulation is consistent with the model that dedifferentiated phenotypes of cancer cells, arising from EMT or other mechanisms, confer tumor aggressiveness, therapeutic resistance, and worse patient outcomes⁷⁴⁻⁷⁶. These expression changes suggest greater plasticity of tumors in the high editing group and may help explain the association observed between high RNA editing load and worse overall survival.

Another class of strongly overexpressed genes in the group with high editing load consists of those involved in type I interferon signaling pathway and other immune processes, such as chronic inflammatory response and negative regulation of T cell proliferation (FIG. 5G). These processes may signal the potential presence of an immunosuppressive tumor microenvironment and T cell exhaustion, which would promote tumor progression and resistance to treatment^{67,77,78}. Thus, these immune-related changes may also contribute to the poorer prognosis of the high editing group.

### DISCUSSION

We conducted the first global study of RNA editing in single cells from cancer. Using scRNA-seq data from lung adenocarcinomas, we observed that cancer cells exhibited an elevated editing trend compared to control epithelial cells. This is in stark contrast to the hypoediting trend observed in other cell types of tumor origin. Thus, our data support that the previously reported heightened editing levels in bulk LUAD tumors primarily reflect editing changes in cancer cells. These results motivated us to examine the relationship between cancer-associated editing and other cancer-intrinsic features including interferon response and tumor mutation burden, for which we confirmed statistically significant correlations. Importantly, despite these correlations, RNA editing load in tumors constitutes the best predictor for patient survival among the three types of features. Our analysis highlights that RNA editing serves as a source of RNA mutations that may have close implications in cancer processes⁷⁹.

The observed negative correlation between editing levels and ISG expression profiles in cancer cells are in line with the existing model where increased editing levels in cancer cells cause repressed interferon production and sustained cell growth²⁶. As a mechanism to avoid mistakenly inducing an innate immune response and halting cell growth. ADAR-catalyzed inosines mark endogenous dsRNAs as self RNAs²²⁻²⁵. The resulting ADAR dependence in certain cancer cell lines, patient-derived xenografts, and mouse models indicates that ADAR acts as an immune checkpoint in cancer with important clinical implications¹⁴⁻¹⁷. However, beyond previous negative associations between editing and general tumor inflammation based on combined TCGA cancer types¹⁴, this relationship had not been demonstrated at the single cell level. Using scRNA-seq data, our findings provide additional support for the hypothesis that RNA editing suppresses the innate immune response in cancer cells of NSCLC. Note that hypoediting in non-epithelial cells of tumors is another interesting observation, the implications of which needs to be further investigated.

Our analysis of RNA editing across samples collected at different treatment stages revealed that PD tumors that developed resistance to targeted therapy had higher editing levels than regressing or treatment-naïve tumors. Genes harboring higher editing in PD are enriched in pathways with close relevance to cancer progression, including immune- and EMT-related pathways. It is possible that these higher editing levels constitute another resistance mechanism of repressing the immune response. Consistent with this hypothesis, we observed a significant overlap between RNA editing sites associated with drug sensitivity and those differentially edited between PD and RD samples. Although a functional relationship is yet to be established, the data indicate that altered editing may contribute to drug resistance and disease progression.

Our results suggest that NK cells may have close relevance to RNA editing-mediated immune response in tumors. NK cells are cytotoxic innate immune cells that can prevent tumor progression. Their antitumor activity can be activated inherently without requiring specific antigen presentation. Thus, NK cells are increasingly recognized as potential candidates to facilitate cancer immunotherapy⁸⁰. We observed that the editing levels of cancer cells negatively correlated with the number of NK cells in the tumor. This negative correlation, together with the observation of heightened editing in cancer cells, points to the possibility that elevated editing in cancer cells contributes to their evasion from NK cell destruction. Future studies need to be carried out to substantiate such a causal relationship. In addition, whether RNA editing may affect the cytotoxicity or IFN production of NK cells needs to be investigated.

Determining cell type specificity of tumor editing changes and delineating the relationship between cancer editing and NK infiltration were among findings enabled by single cell sequencing technology. Through scRNA-seq analysis, editing events can be identified within individual cells. This advantage of scRNA-seq allowed us to characterize tumor RNA editing patterns at a much finer resolution than afforded by bulk RNA-seq used in previous studies. The method used to identify RNA editing sites in scRNA-seq is largely similar to those for bulk RNA-seq. Nonetheless, since the read coverage in each cell is relatively low, fewer RNA editing events may be detected in single cells than in bulk RNA-seq. Thus, for certain applications, a pseudobulk approach may be adopted. For example, in detecting differential editing between tumor and non-malignant samples, we took the pseudobulk approach by pooling reads from all cells of each cell type to enhance the total read coverage since this analysis does not require a single-cell resolution. In the future, single-cell-based differential editing method may be developed if variations between individual cells are of interest. Lastly, identification of editing sites in scRNA-seq data depends on the specific protocol used for data generation. Smart-seq2 or other full-length scRNA-seq methods may be preferred over methods that capture only the 5′ or 3′ ends of the transcripts to identify RNA editing sites in full-length mRNAs.

We observed a positive correlation between RNA editing load and TMB in cancer cells and bulk tumors, linking together the two distinct sources of mutations observed in the RNA. Importantly, we show that this relationship is specific to cancer cells of tumors and may involve editing of specific genes in DNA damage response. The reason behind this apparent correlation remains unclear. Since TMB is associated with cytolytic activity in certain cancer types like LUAD⁶⁹, higher TMB may prompt IFN-γ secretion by cytotoxic T cells^{81,82} through increased neoantigen load. As a result, IFN-γ may induce ADAR p150 expression⁸³ in cancer cells. However, there was no consistent change of ADAR or p150 expression in the scRNA-seq data or bulk tumors (FIG. S10 in Chan et al.). Alternatively, RNA editing changes may directly or indirectly impact somatic mutation burden in cancer cells. It is also possible that TMB and RNA editing load are independent of each other, but both related to an unknown causal factor.

We also note that RNA editing load was defined differently in the single cell and bulk tumor analyses to more accurately reflect RNA editing of cancer cells from each dataset. Since editing levels can be measured specifically within cancer cells in the scRNA-seq data, we calculated RNA editing load as the mean editing level over all sites of cancer cells. However, isolating cancer cell editing levels directly from bulk RNA-seq of tumors, comprising multiple cell types, is not possible. Consequently, for the bulk RNA-seq data, we based RNA editing load on tumor-increased sites that were identified as differentially edited between tumors and matched non-malignant samples. In situations where no non-malignant samples are available, all editing sites could be used to calculate RNA editing load of bulk tumors (FIG. S11 in Chan et al.).

Our analyses showed that the RNA editing load was a stronger predictor of patient survival than TMB and expression levels of interferon response signatures. Importantly, a differential expression analysis between tumors with high and low RNA editing load uncovered many genes with close relevance to cancer. One prominent category of genes with increased expression in the group with a high RNA editing load consists of those related to cell cycle. Cell cycle regulation is a key aspect of cancer, which may bypass DNA damage checkpoints and lead to increased TMB^{72,73}. Thus, the above data provide support for the functional relevance of RNA editing in cancer.

Together, our results showed that, as a source of RNA mutations, RNA editing is an important aspect of cancer. The amount of RNA editing in cancer cells, namely RNA editing load, correlates with other cancer-intrinsic features. RNA editing load is useful as a predictor of patient survival in LUAD and as a predictor for response to cancer therapies.

### REFERENCES

1. Nishikura, K. A-to-I editing of coding and non-coding RNAs by ADARs. Nat. Rev. Mol. Cell Biol. 17, 83-96 (2016).
2. Kung, C.-P., Maggi, L. B. & Weber, J. D. The Role of RNA Editing in Cancer Development and Metabolic Disorders. Front. Endocrinol. (Lausanne). 9, 762 (2018).
3. Xu, X., Wang, Y. & Liang, H. The role of A-to-I RNA editing in cancer development. Curr. Opin. Genet. Dev. 48, 51-56 (2018).
4. Erdmann, E. A., Mahapatra, A., Mukherjee, P., Yang, B. & Hundley, H. A. To protect and modify double-stranded RNA—the critical roles of ADARs in development, immunity and oncogenesis. Critical Reviews in Biochemistry and Molecular Biology 56, 54-87 (2021).
5. Song, B., Shiromoto, Y., Minakuchi, M. & Nishikura, K. The role of RNA editing enzyme ADAR1 in human disease. Wiley Interdisciplinary Reviews: RNA (2021). doi:10.1002/wrna.1665
6. Chen, L. et al. Recoding RNA editing of AZIN1 predisposes to hepatocellular carcinoma. Nat. Med. 19, 209-216 (2013).
7. Hu. X. et al. RNA editing of AZIN1 induces the malignant progression of non-small-cell lung cancers. Tumor Biol. 39, (2017).
8. Zhang, M. et al. RNA editing derived epitopes function as cancer antigens to elicit immune responses. Nat. Commun. 9, 3919 (2018).
9. Peng, X. et al. A-to-I RNA Editing Contributes to Proteomic Diversity in Cancer. Cancer Cell 33, 1-12 (2018).
10. Chan, T. W. et al. RNA editing in cancer impacts mRNA abundance in immune response pathways. Genome Biol. 21, 268 (2020).
11. Amin, E. M. et al. The RNA-editing enzyme ADAR promotes lung adenocarcinoma migration and invasion by stabilizing FAK. Sci. Signal. 10, (2017).
12. Marceca, G. P. et al. Detecting and characterizing a-to-i microrna editing in cancer. Cancers 13, 1699 (2021).
13. Brummer, A., Yang, Y., Chan, T. W. & Xiao, X. Structure-mediated modulation of mRNA abundance by A-to-I editing. Nat. Commun. 8, 1-12 (2017).
14. Ishizuka, J. J. et al. Loss of ADAR1 in tumours overcomes resistance to immune checkpoint blockade. Nature 565, 43-48 (2019).
15. Mehdipour, P. et al. Epigenetic therapy induces transcription of inverted SINEs and ADAR1 dependency. Nature 588, 169-173 (2020).
16. Gannon, H. S. et al. Identification of ADAR1 adenosine deaminase dependency in a subset of cancer cells. Nat. Commun. 9. 5450 (2018).
17. Liu. H. et al. Tumor-derived IFN triggers chronic pathway agonism and sensitivity to ADAR loss. Nat. Med. 25, 95-102 (2019).
18. Han, L. et al. The Genomic Landscape and Clinical Relevance of A-to-I RNA Editing in Human Cancers. Cancer Cell 28, 515-528 (2015).
19. Paz-Yaacov, N. et al. Elevated RNA Editing Activity Is a Major Contributor to Transcriptomic Diversity in Tumors. Cell Rep. 13, 267-276 (2015).
20. Fumagalli, D. et al. Principles Governing A-to-I RNA Editing in the Breast Cancer Transcriptome. Cell Rep. 13. 277-289 (2015).
21. Lamers, M. M., van den Hoogen, B. G. & Haagmans, B. L. ADAR1: Editor-in-Chief of Cytoplasmic Innate Immunity. Frontiers in immunology 10, 1763 (2019).
22. Ahmad. S., Mu, X. & Hur, S. The role of ma editing in the immune response, in Methods in Molecular Biology 2181. 287-307 (Humana Press Inc., 2021).
23. Ahmad, S. et al. Breaching Self-Tolerance to Alu Duplex RNA Underlies MDA5-Mediated Inflammation. Cell 172, 797-810.e13 (2018).
24. Chung, H. et al. Human ADAR1 Prevents Endogenous RNA from Triggering Translational Shutdown. Cell 172, 811-824.e14 (2018).
25. Hartner, J. C., Walkley, C. R., Lu, J. & Orkin, S. H. ADAR1 is essential for the maintenance of hematopoiesis and suppression of interferon signaling. Nat. Immunol. 10, 109-115 (2009).
26. Xu. L.-D. & Ohman, M. ADAR1 Editing and its Role in Cancer. Genes (Basel). 10, (2019).
27. Dentro, S. C. et al. Characterizing genetic intra-tumor heterogeneity across 2,658 human cancer genomes. Cell 184, 2239-2254.e39 (2021).
28. Bhandari. V. et al. Molecular landmarks of tumor hypoxia across cancer types. Nat. Genet. 51, 308-318 (2019).
29. Maynard, A. et al. Therapy-Induced Evolution of Human Lung Cancer Revealed by Single-Cell RNA Sequencing. Cell 182, 1232-1251.e22 (2020).
30. Dobin, A. et al. STAR: Ultrafast universal RNA-seq aligner. Bioinformatics 29, 15-21 (2013).
31. Hao, Y. et al. Integrated analysis of multimodal single-cell data. Cell 184, 3573-3587.e29 (2021).
32. Hafemeister, C. & Satija, R. Normalization and variance stabilization of single-cell RNA-seq data using regularized negative binomial regression. Genome Biol. 20, 296 (2019).
33. Travaglini, K. J. et al. A molecular cell atlas of the human lung from single-cell RNA sequencing. Nature 587, 619-625 (2020).
34. inferCNV of the Trinity CTAT Project. Available at: https://github.com/broadinstitute/inferCNV. (Accessed: 23 Jul. 2021)
35. Genomic Data Commons. Available at: https://portal.gdc.cancer.gov/. (Accessed: 10 Mar. 2020)
36. Kim, D., Paggi, J. M., Park, C., Bennett, C. & Salzberg, S. L. Graph-based genome alignment and genotyping with HISAT2 and HISAT-genotype. Nat. Biotechnol. 37, 907-915 (2019).
37. Tran, S. S. et al. Widespread RNA editing dysregulation in brains from autistic individuals. Nat. Neurosci. 22, 25-36 (2019).
38. Porath, H. T., Carmi, S. & Levanon, E. Y. A genome-wide map of hyperedited RNA reveals numerous new sites. Nat. Commun. 5, 4726 (2014).
39. Bahn, J. H. et al. Accurate identification of A-to-I RNA editing in human by transcriptome sequencing. Genome Res. 22, 142-150 (2012).
40. Lee, J., Ang, J. K. & Xiao, X. Analysis and design of RNA sequencing experiments for identifying RNA editing and other single-nucleotide variants. RNA 19, 725-732 (2013).
41. Mansi, L. et al. REDIportal: Millions of novel A-to-I RNA editing events from thousands of RNAseq experiments. Nucleic Acids Res. 49, D1012-D1019 (2021).
42. Fu, L. et al. RNA editing of SLC22A3 drives early tumor invasion and metastasis in familial esophageal cancer. PNAS 114, 4631-4640 (2017).
43. Dong, X. et al. CDK13 RNA Over-Editing mediated by ADAR1 associates with poor prognosis of hepatocellular carcinoma patients. Cell. Physiol. Biochem. 47, 2602-2612 (2018).
44. Chen, Y. Bin et al. ADAR2 functions as a tumor suppressor via editing IGFBP7 in esophageal squamous cell carcinoma. Int. J. Oncol. 50, 622-630 (2017).
45. Jiang. Q. et al. Hyper-Editing of Cell-Cycle Regulatory and Tumor Suppressor RNA Promotes Malignant Progenitor Propagation. Cancer Cell 35, 81-94.e7 (2019).
46. Beghini, A. et al. RNA hyperediting and alternative splicing of hematopoietic cell phosphatase (PTPN6) gene in acute myeloid leukemia. Hum. Mol. Genet. 9. 2297-2304 (2000).
47. Levanon, E. Y. et al. Evolutionarily conserved human targets of adenosine to inosine RNA editing. Nucleic Acids Res. 33, 1162-1168 (2005).
48. Eisenberg, E. et al. Identification of RNA editing sites in the SNP database. Nucleic Acids Res. 33, 4612-4617 (2005).
49. Collisson, E. A. et al. Comprehensive molecular profiling of lung adenocarcinoma. Nature 511, 543-550 (2014).
50. Tran, S. S., Zhou, Q. & Xiao, X. Statistical inference of differential RNA-editing sites from RNA-sequencing data by hierarchical modeling. Bioinformatics 36, 2796-2804 (2020).
51. Finotello, F. et al. Molecular and pharmacological modulators of the tumor immune contexture revealed by deconvolution of RNA-seq data. Genome Med. 11, 34 (2019).
52. Li, T. et al. TIMER2.0 for analysis of tumor-infiltrating immune cells. Nucleic Acids Res. 48, W509-W514 (2020).
53. Wang, C. et al. Identification of A-to-I RNA editing profiles and their clinical relevance in lung adenocarcinoma. Sci. China Life Sci. 1-14 (2021). doi:10.1007/s11427-020-1928-0
54. Wu, F. et al. Single-cell profiling of tumor heterogeneity and the microenvironment in advanced non-small cell lung cancer. Nat. Commun. 12, 1-11 (2021).
55. Lambrechts, D. et al. Phenotype molding of stromal cells in the lung tumor microenvironment. Nat. Med 24, 1277-1289 (2018).
56. Fu, J., Wang, X. & Yue, Q. Functional loss of TAGLN inhibits tumor growth and increases chemosensitivity of non-small cell lung cancer. Biochem.
   Biophys. Res. Commun. 529. 1086-1093 (2020).
57. Elsafadi, M. et al. Transgelin is a poor prognostic factor associated with advanced colorectal cancer (CRC) stage promoting tumor growth and migration in a TGFβ-dependent manner. Cell Death Dis. 11, 1-13 (2020).
58. Li, P., Lan, P., Liu, S., Wang, Y. & Liu, P. Cell Polarity Protein Pals1-Associated Tight Junction Expression Is a Favorable Prognostic Marker in Clear Cell Renal Cell Carcinoma. Front. Genet. 11, 931 (2020).
59. Pruski, M. et al. The ciliary GTPase Arl13b regulates cell migration and cell cycle progression. Cell Adhes. Migr. 10, 393-405 (2016).
60. Ruan. H. et al. GPEdit: the genetic and pharmacogenomic landscape of A-to-I RNA editing in cancers. Nucleic Acids Res. 50, D1231-D1237 (2022).
61. Liddicoat, B. J. et al. RNA editing by ADAR1 prevents MDA5 sensing of endogenous dsRNA as nonself. Science (80-.). 349, 1115-1120 (2015).
62. Walkley, C. R. & Li, J. B. Rewriting the transcriptome: adenosine-to-inosine RNA editing by ADARs. Genome Biol. 18, 1-13 (2017).
63. Vitali, P. & Scadden, A. D. J. Double-stranded RNAs containing multiple IU pairs are sufficient to suppress interferon induction and apoptosis. Nat. Struct. Mol. Biol. 17. 1043-1050 (2010).
64. Benci, J. L. et al. Opposing Functions of Interferon Coordinate Adaptive and Innate Immune Responses to Cancer Immune Checkpoint Blockade. Cell 178, 933-948.e14 (2019).
65. Cheon, H. et al. IFNβ-dependent increases in STAT1, STAT2, and IRF9 mediate resistance to viruses and DNA damage. EMBO J. 32, 2751-2763 (2013).
66. Cheon, H. J. & Stark, G. R. Unphosphorylated STAT1 prolongs the expression of interferon-induced immune regulatory genes. Proc. Natl. Acad. Sci. U.S.A. 106, 9373-9378 (2009).
67. Budhwani, M., Mazzieri, R. & Dolcetti, R. Plasticity of Type I Interferon-Mediated Responses in Cancer Therapy: From Anti-tumor Immunity to Resistance. Front. Oncol. 8, 322 (2018).
68. Thorsson, V. et al. The Immune Landscape of Cancer. Immunity 48, 812-830.e14 (2018).
69. Rooney, M. S., Shukla, S. A., Wu, C. J., Getz, G. & Hacohen, N. Molecular and genetic properties of tumors associated with local immune cytolytic activity. Cell 160, 48-61 (2015).
70. Wu, Y. et al. The Predictive Value of Tumor Mutation Burden on Efficacy of Immune Checkpoint Inhibitors in Cancers: A Systematic Review and Meta-Analysis. Frontiers in Oncology 9, 1161 (2019).
71. Rizvi. N. A. et al. Mutational landscape determines sensitivity to PD-1 blockade in non-small cell lung cancer. Science (80-.). 348, 124-128 (2015).
72. Matthews, H. K., Bertoli, C. & de Bruin, R. A. M. Cell cycle control in cancer. Nature Reviews Molecular Cell Biology 23, 74-88 (2022).
73. Otto, T. & Sicinski, P. Cell cycle proteins as promising targets in cancer therapy. Nature Reviews Cancer 17, 93-115 (2017).
74. Friedmann-Morvinski, D. & Verma, I. M. Dedifferentiation and reprogramming: origins of cancer stem cells. EMBO Rep. 15, 244-253 (2014).
75. Yuan. S., Norgard, R. J. & Stanger, B. Z. Cellular Plasticity in Cancer. Cancer Discov. 9, 837-851 (2019).
76. Boumahdi, S. & de Sauvage, F. J. The great escape: tumour cell plasticity in resistance to targeted therapy. Nat. Rev. Drug Discov. 2019 191 19, 39-56 (2019).
77. Zhao. H. et al. Inflammation and tumor progression: signaling pathways and targeted intervention. Signal Transduction and Targeted Therapy 6, 1-46 (2021).
78. Boukhaled, G. M., Harding, S. & Brooks, D. G. Opposing Roles of Type I Interferons in Cancer Immunity. https: doi.org/10.1146/annurev-pathol-031920-093932 16, 167-198 (2021).
79. Ben-Aroya, S. & Levanon, E. Y. A-to-I RNA Editing: An Overlooked Source of Cancer Mutations. Cancer Cell 33, 789-790 (2018).
80. St-Pierre, F., Bhatia, S. & Chandra, S. Harnessing natural killer cells in cancer immunotherapy: A review of mechanisms and novel therapies. Cancers 13, 1988 (2021).
81. Bhat, P., Leggatt. G., Waterhouse, N. & Frazer, I. H. Interferon-γ derived from cytotoxic lymphocytes directly enhances their motility and cytotoxicity. Cell Death Dis. 8, e2836 (2017).
82. Castro, F., Cardoso, A. P., Gongalves, R. M., Serre, K. & Oliveira, M. J. Interferon-gamma at the crossroads of tumor immune surveillance or evasion. Frontiers in Immunology 9, 847 (2018).
83. Patterson, J. B., Thomis, D. C., Hans, S. L. & Samuel, C. E. Mechanism of Interferon Action: Double-Stranded RNA-Specific Adenosine Deaminase from Human Cells Is Inducible by Alpha and Gamma Interferons. Virology 210, 508-511 (1995).

TABLE S1
Recoding sites elevated in cancer cells.

| Editing site | | Protein | Adjusted | Difference in |
|---|---|---|---|---|
| coordinate | Gene | change | p-value* | mean editing |
| chr1: 225974568 | SRP9 | Q49R | 0.0323 | 0.167 |
| chr1: 62628525 | INADL | R1146G | 0.00135 | 0.157 |
| chr11: 117073797 | TAGLN | D23G | 0.00217 | 0.199 |
| chr12: 69160465 | SLC35E3 | S56G | 0.0465 | 1 |
| chr3: 93715457 | ARL13B | S61G | 8.69E−05 | 0.25 |
| chr5: 50037702 | PARP8 | I97M | 0.0184 | 0.983 |

*Adjusted p-value based on comparing editing in cancer cells and non-malignant epithelial cells using REDITs

All publications mentioned herein (e.g., Chan et al., Cancer Res. 2023 Feb. 3; 83(3):374-385, and those disclosed above) are incorporated by reference to disclose and describe aspects, methods and/or materials in connection with the cited publications. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

1. A method of obtaining information useful for predicting patient survival in a patient diagnosed with a cancer, the method comprising:
obtaining a cancerous cell from the patient;
obtaining double stranded RNA from the cancerous cell;
observing double stranded RNA editing in the RNA obtained from the cancerous cell; and
correlating the double stranded RNA editing observed with patient survival such information useful for predicting patient survival in a patient diagnosed with a cancer is obtained.

2. The method of claim 1, wherein the method includes:
obtaining a noncancerous cell from the patient;
obtaining double stranded RNA from the noncancerous cell;
observing double stranded RNA editing in the RNA obtained from the noncancerous cell; and
comparing levels of double stranded RNA editing observed in the cancerous cell with levels of double stranded RNA editing observed in the noncancerous cell.

3. The method of claim 2, wherein an RNA editing load observed in the cancerous cell that is at least 10%, 20% or 30% greater than the editing load observed in the noncancerous cell provides evidence of likely tumor progression and/or resistance to treatment with a chemotherapeutic agent.

4. The method of claim 1, wherein RNA editing is observed in at least one double stranded RNA transcribed from a cathepsin B gene, a small integral membrane protein 14 gene, or an aryl hydrocarbon receptor gene.

5. The method of claim 1, wherein double stranded RNA editing is observed in at least one double stranded RNA selected from: Alu-dsRNA: chr19:9635175-9635484 AluSp_SINE; Alu-dsRNA: chr14:66737078-66737348 AluJb_SINE; Alu-dsRNA: chr21:34806547-34806850 AluSx1_SINE; and Alu-dsRNA: chr7:99960634-99060779 FLAM_C_SINE.

6. The method of claim 1, wherein the cancer cell is obtained from a person diagnosed with a lung cancer or a colorectal cancer.

7. The method of claim 1, wherein the method includes a multiplexed polymerase chain reaction process.

8. The method of claim 1, wherein the double stranded RNA editing observed is correlated with patient response to a therapeutic intervention, tumor mutation burden or cancer innate immunity.

9. The method of claim 1, wherein the method is performed on a single cell.

10. A method of observing double stranded RNA (dsRNA) editing in a human cell comprising:
obtaining dsRNA from one or more human cells;
observing dsRNA editing in the dsRNA obtained from the cell, wherein dsRNA editing is observed in at least one:
dsRNA transcribed from a cathepsin B gene, a small integral membrane protein 14 gene, or an aryl hydrocarbon receptor gene;
dsRNA selected from: Alu-dsRNA: chr19:9635175-9635484 AluSp_SINE; Alu-dsRNA: chr14:66737078-66737348 AluJb_SINE; Alu-dsRNA: chr21:34806547-34806850 AluSx1_SINE; and Alu-dsRNA: chr7:99960634-99060779 FLAM_C_SINE;
and/or dsRNAs encoding a SRP9 polypeptide, a INADL polypeptide, a TAGLN polypeptide, a SLC35E3 polypeptide, a ARL13B polypeptide or a PARP8 polypeptide.

11. The method of claim 10, wherein the dsRNA editing observed results in at least one alteration in a polypeptide sequence selected from:
Q49R in the SRP9 polypeptide;
R1146G in the INADL polypeptide;
D23G in the TAGLN polypeptide;
S56G in the SLC35E3 polypeptide;
S61G in the ARL13B polypeptide; or
I97M in the PARP8 polypeptide.

12. The method of claim 11, wherein the dsRNA editing observed results in at least two, three or four alterations in a polypeptide sequence selected from:
Q49R in the SRP9 polypeptide;
R1146G in the INADL polypeptide;
D23G in the TAGLN polypeptide;
S56G in the SLC35E3 polypeptide;
S61G in the ARL13B polypeptide; or
I97M in the PARP8 polypeptide.

13. The method of claim 10, wherein the method is performed on a single cell.

14. The method of claim 10, wherein the cell is a cancer cell.

15. The method of claim 14, wherein the cancer cell is obtained from a person diagnosed with a lung cancer or a colorectal cancer.

16. The method of claim 10, wherein the dsRNA editing observed is correlated with patient response to a therapeutic intervention, tumor mutation burden or cancer innate immunity.

17. The method of claim 10, wherein the method includes observing amounts/load of edited dsRNA in the cell.
